(19) United States
(12) Patent Application Publication
(10) Pub. No.: US 2023/0184585 A1
(43) Pub. Date: Jun. 15, 2023

(54) ENVIRONMENTAL DATA ANALYSIS AND GENERATION OF NOTIFICATION USING MOBILE DEVICE

(71) Applicant: International Business Machines Corporation, Armonk, NY (US)

(72) Inventors: Nimra Tariq, Leesburg, VA (US); Jacob Greenleaf, Lexington, NC (US); Caroline Li, Johns Creek, GA (US); Zachary A. Silverstein, Georgetown, TX (US)

(21) Appl. No.: 17/644,262

(22) Filed: Dec. 14, 2021

(51) Int. Cl. G01J 1/44; H04W 4/029; G08B 21/18; G08B 21/02; G01S 19/14

(52) U.S. Cl. CPC G01J 1/44; H04W 4/029; G08B 21/182; G08B 21/02; G01S 19/14; G16H 10/65

(57) ABSTRACT

A mobile device can be used for detecting and communicating to a user at a location, sunlight exposure in relation to the user at the location. Sunlight intensity data at a location of a mobile device is determined, in response to receiving at the computer, GPS (Global Positioning System) data for the mobile device. The sunlight intensity data and the GPS data is analyzed to generate an estimate of sunlight exposure for a user using the mobile device at the location. A level of sunlight exposure for the user is determined, based on the estimate of sunlight exposure for the user, and using the mobile device, the level of sunlight exposure to the user is communicated to the user.

### BACKGROUND

The present disclosure relates to receiving and analyzing, using a computer, environmental data at a location of a user, and communicating a notification using a mobile device to the user.

Environmental hazards while outdoors can be a concern when people are outside. For example, people can often be outside using their mobile phone, but don't realize their exposure to sunlight. Although sun exposure can have positive effects, spending too much time in the sun can have negatively impact a person's health, that is, can be harmful. Whether as a result of outside physical activity or leisure time outside, too much sun exposure can put a person at risk for skin damage (e.g., nonmelanoma and melanoma skin cancer, clumps of skin cells, wrinkles, hives, skin irritation, sunburn, etc.), a lowered immune system, eye injuries, dehydration, and overheating.

### SUMMARY

The present disclosure recognizes the shortcomings and problems associated with current techniques communicating sunlight exposure to a user. For instance, people often carry their mobile devices, and thus according to the present invention, mobile devices provide an accessible way to gather data surrounding exposure of a user. For example, in one embodiment of the present invention, a system and method can include detecting and communicating a notification about sunlight exposure to a user by using a mobile device to detect sunlight intensity and communicate an alert or notification regarding the sunlight intensity to a user.

In one example, a system and method can utilizes GPS location, and photosensitive sensors and a forward and backwards facing camera to derive sunlight exposure metrics and risks for a user of the mobile device, and as a result, communicate using the mobile device a notification or alert regarding the sunlight exposure to the user.

In an aspect according to the present invention, a computer-implemented method for detecting and communicating to a user at a location, sunlight exposure in relation to the user at the location. The method includes determining, at a computer, sunlight intensity data at a location of a mobile device, in response to receiving at the computer, GPS (Global Positioning System) data for the mobile device. The method includes analyzing the sunlight intensity data and the GPS data to generate an estimate of sunlight exposure for a user using the mobile device at the location. The method includes determining a level of sunlight exposure for the user, based on the estimate of sunlight exposure for the user, and communicating, using the mobile device, the level of sunlight exposure to the user.

In a related aspect, the communicating includes sending a notification to the mobile device communicating the level of sunlight exposure to the user.

In a related aspect, the communicating includes displaying the level of sunlight exposure on a display of the mobile device.

In a related aspect, the communicating includes generating an audible notification of the level of sunlight exposure using the mobile device.

In a related aspect, the sunlight intensity data is determined using a camera of the mobile device to estimate the sunlight intensity.

In a related aspect, the computer is part of the mobile device.

In a related aspect, the computer is at least part of a remote device communicating with the mobile device.

In a related aspect, the method includes determining a sunlight exposure threshold for the user based on an exposure formula for safety; and using the analysis of the sunlight intensity, determining when the sunlight exposure threshold is met, and in response to the sunlight exposure being met, communicating a warning to the user which include the communicating of the level of sunlight exposure.

In a related aspect, the method further including accessing a historical data corpus for the user which includes a medical history; determining a sunlight exposure threshold for the user based on the medical history; using the analysis of the sunlight intensity data, determining when the sunlight exposure threshold is met; and communicating an alert to the user which includes the communicating of the level of sunlight exposure, in response to the sunlight exposure being met.

In a related aspect, the method further including generating a model, using the computer wherein the model includes the following; updating the sunlight intensity data;
updating the analyzing of the sunlight intensity data; updating the determining of the level of the sunlight exposure; and updating the communicating of the level of the sunlight exposure.

On a related aspect, the method further including iteratively generating the model to produce updated models.

In another aspect according to the present invention, a system uses a mobile device for detecting and communicating to a user at a location, sunlight exposure in relation to the user at the location. The system comprises: a computer system comprising; a computer processor, a computer-readable storage medium, and program instructions stored on the computer-readable storage medium being executable by the processor, to cause the computer system to perform the following functions to; determining, at a computer, sunlight intensity data at a location of a mobile device, in response to receiving at the computer, GPS (Global Positioning System) data for the mobile device; analyzing the sunlight intensity data and the GPS data to generate an estimate of sunlight exposure for a user using the mobile device at the location; determining a level of sunlight exposure for the user, based on the estimate of sunlight exposure for the user; and communicating, using the mobile device, the level of sunlight exposure to the user.

In a related aspect, the communicating includes sending a notification to the mobile device communicating the level of sunlight exposure to the user.

In a related aspect, the communicating includes displaying the level of sunlight exposure on a display of the mobile device.

In a related aspect, the communicating includes generating an audible notification of the level of sunlight exposure using the mobile device.

In a related aspect, the sunlight intensity data is determined using a camera of the mobile device to estimate the sunlight intensity.

In a related aspect, the computer is part of the mobile device.

In a related aspect, the computer is at least part of a remote device communicating with the mobile device.

In a related aspect, the system further comprising: determining a sunlight exposure threshold for the user based on an exposure formula for safety; and using the analysis of the sunlight intensity, determining when the sunlight exposure threshold is met, and in response to the sunlight exposure being met, communicating a warning to the user which include the communicating of the level of sunlight exposure.

In another embodiment according to the present invention, a computer program product uses a mobile device for detecting and communicating to a user at a location, sunlight exposure in relation to the user at the location. The computer program product comprising a computer readable storage medium having program instructions embodied therewith, the program instructions executable by a computer to cause the computer to perform functions, by the computer, comprising the functions to; determining, at a computer, sunlight intensity data at a location of a mobile device, in response to receiving at the computer, GPS (Global Positioning System) data for the mobile device; analyzing the sunlight intensity data and the GPS data to generate an estimate of sunlight exposure for a user using the mobile device at the location; determining a level of sunlight exposure for the user, based on the estimate of sunlight exposure for the user; and communicating, using the mobile device, the level of sunlight exposure to the user.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. The drawings are discussed forthwith below.

FIG. 1 is a schematic block diagram illustrating an overview of a system, system features or components, and methodology for detecting and communicating to a user at a location, sunlight exposure in relation to the user at the location, according to an embodiment of the present disclosure.

FIG. 2 is a flow chart illustrating a method, implemented using the system shown in FIG. 1, for detecting and communicating to a user at a location, sunlight exposure in relation to the user at the location, according to an embodiment of the present disclosure.

FIG. 3 is a functional schematic block diagram showing a series of operations and functional methodologies, for instructional purposes illustrating functional features of the present disclosure associated with the embodiments shown in the FIGS., which can be implemented, at least in part, in coordination with the system shown in FIG. 1, for detecting and communicating to a user at a location, sunlight exposure in relation to the user at the location.

FIG. 4A is a flow chart illustrating another method, which continues from the flow chart of FIG. 2, for detecting and communicating to a user at a location, sunlight exposure in relation to the user at the location, according to an embodiment of the present disclosure.

FIG. 4B is a flow chart illustrating another method, which continues from the flow chart of FIG. 2, for detecting and communicating to a user at a location, sunlight exposure in relation to the user at the location, including an AI model and data analysis, according to an embodiment of the present disclosure.

FIG. 5 is a flow chart illustrating another method according to an embodiment of the present invention, for detecting and communicating to a user at a location, sunlight exposure in relation to the user at the location, according to an embodiment of the present disclosure.

FIG. 6 is a schematic block diagram depicting a computer system according to an embodiment of the disclosure which may be incorporated, all or in part, in one or more computers or devices shown in FIG. 1, and cooperates with the systems and methods shown in the FIGS.

FIG. 7 is a block diagram depicting a cloud computing environment according to an embodiment of the present invention.

FIG. 8 is a block diagram depicting abstraction model layers according to an embodiment of the present invention.

### DETAILED DESCRIPTION

The following description with reference to the accompanying drawings is provided to assist in a comprehensive understanding of exemplary embodiments of the invention as defined by the claims and their equivalents. The description includes various specific details to assist in that understanding, but these are to be regarded as merely exemplary, and assist in providing clarity and conciseness. Accordingly, those of ordinary skill in the art will recognize that various changes and modifications of the embodiments described herein can be made without departing from the scope and spirit of the invention. In addition, descriptions of well-known functions and constructions may be omitted.

The terms and words used in the following description and claims are not limited to the bibliographical meanings, but are merely used to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

### EMBODIMENTS AND EXAMPLES

Embodiments and figures of the present disclosure may have the same or similar components as other embodiments. Such figures and descriptions of illustrate and explain further examples and embodiments according to the present disclosure.

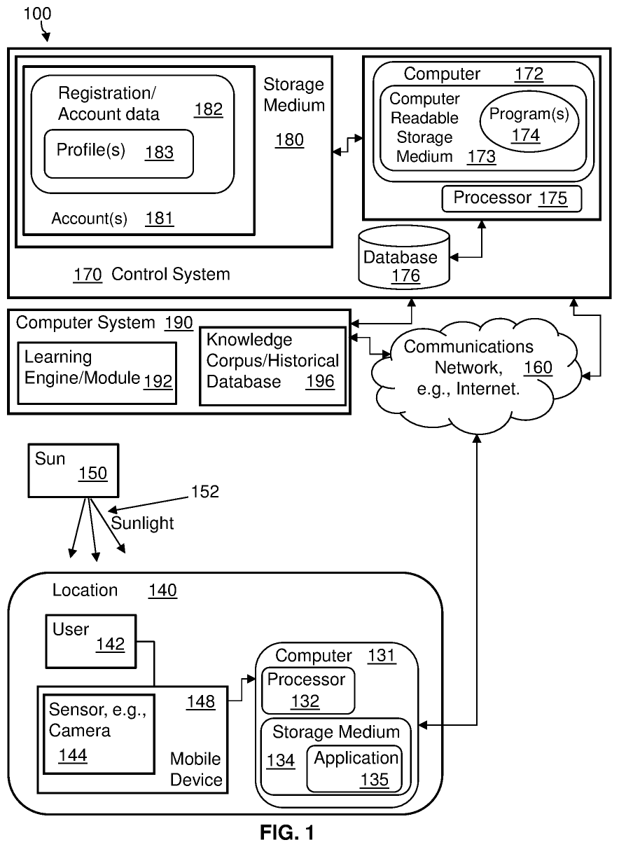

Referring to FIGS. 1, 2 and 3, according to embodiments of the present disclosure, a computer-implemented method 200 for detecting and communicating to a user 142 at a location 140, sunlight exposure 152 from the sun 150 in relation to the user at the location includes features described below. Embodiments of the present disclosure include operational actions and/or procedures. The computer-implemented method 200 includes a series of operational blocks for implementing an embodiment according to the present disclosure which can include the system shown in FIG. 1. The operational blocks of the methods and systems according to the present disclosure can include techniques, mechanism, modules, and the like for implementing the functions of the operations in accordance with the present disclosure.

The method 200 includes determining, at a computer 131, sunlight intensity data 308 at a location 140 of a mobile device 148, in response to receiving at the computer, GPS (Global Positioning System) data 312 for the mobile device, as in block 204. In another example, historical data 314 can also be received at the computer.

The method 200 includes analyzing 316 the sunlight intensity data 308 and the GPS data to generate an estimate of sunlight exposure 320 for a user using the mobile device at the location, as in block 208. For example, the estimate can be generated using input from a sensor, such as a camera 144 of the mobile device.

The method includes determining a level of sunlight exposure 324 for the user, based on the estimate of sunlight exposure for the user, as in block 212.

When a threshold for sunlight exposure for the user is not met, at block 216, the method returns to block 212. When a threshold for sunlight exposure for the user is met, at block 216, the method proceeds to block 220.

The method includes communicating 328, using the mobile device, the level of sunlight exposure to the user, as in block 220.

In one example, the communicating can include sending a notification to the mobile device communicating the level of sunlight exposure to the user.

In another example, the communicating includes displaying the level of sunlight exposure on a display of the mobile device. In another example, the communicating includes generating an audible notification of the level of sunlight exposure using the mobile device. In another example, the sunlight intensity data can be determined using a camera 144 of the mobile device to estimate the sunlight intensity. In another example, the computer is part of the mobile device. In another example, the computer is at least part of a remote device communicating with the mobile device.

In another example, the method can include determining a sunlight exposure threshold for the user based on an exposure formula for safety; and using the analysis of the sunlight intensity, determining when the sunlight exposure threshold is met, and in response to the sunlight exposure being met, communicating a warning to the user which include the communicating of the level of sunlight exposure.

In another example, the method includes accessing a historical data corpus for the user which includes a medical history; determining a sunlight exposure threshold for the user based on the medical history; using the analysis of the sunlight intensity data, determining when the sunlight exposure threshold is met; and communicating an alert to the user which includes the communicating of the level of sunlight exposure, in response to the sunlight exposure being met.

In another example, the method can include generating a model, using the computer wherein the model includes the following. The method includes updating the sunlight intensity data; updating the analyzing of the sunlight intensity data; and updating the determining of the level of the sunlight exposure; and updating the communicating of the level of the sunlight exposure. In another example, the method includes iteratively generating the model to produce updated models.

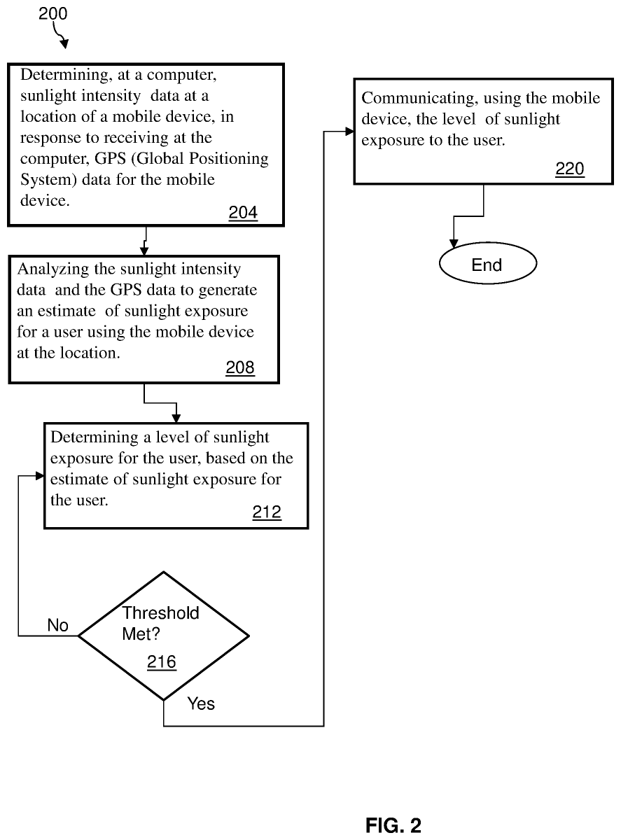

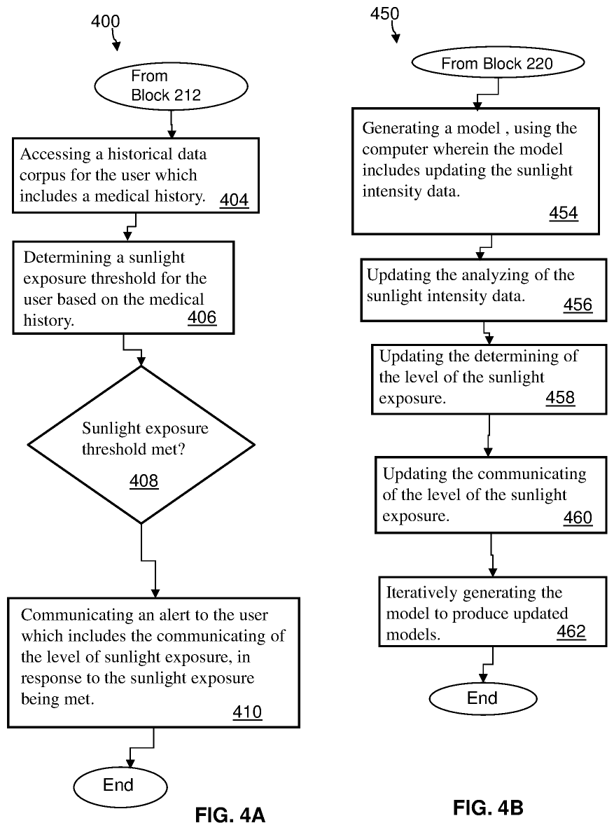

Referring to FIG. 4A, in another embodiment according to the present disclosure, a method 400 continues from block 212 of the method 200 shown in FIG. 2, includes accessing a historical data corpus for the user which includes a medical history, as in block 404. The method includes determining a sunlight exposure threshold for the user based on the medical history, as in block 406. When the sunlight exposure threshold is not met, at block 408, the method returns to block 406. When the sunlight exposure threshold is met, at block 408, the method proceeds to block 410. The method includes communicating an alert to the user which includes the communicating of the level of sunlight exposure, in response to the sunlight exposure being met, as in block 410.

Referring to FIG. 4B, in another embodiment according to the present disclosure, a method 450 continues from block 220 of the method 200 shown in FIG. 2, includes Generating a model, using the computer wherein the model includes updating the sunlight intensity data, as in block 454. The method 450 includes Updating the analyzing of the sunlight intensity data, as in block 456. The method 450 includes updating the determining of the level of the sunlight exposure, as in block 458. The method 450 includes updating the communicating of the level of the sunlight exposure, as in block 460. The method includes iteratively generating the model to produce updated models, as in block 462.

The computer 131 can be integral to or communicating with the robotic device 148 in a device 130. A computer 190 remote from the device 148 can electronically communicate, in all or in part, with the computer 172 as part of the control system 170. The control system can include the computer 172 having a computer readable storage medium 173 which can store one or more programs 174, and a processor 175 for executing program instructions. The control system can also include a storage medium which can include registration and/or account data 182 and profiles 183 of users or entities (such entities can include robotic entities) as part of user accounts 181. User accounts 181 can be stored on a storage medium 180 which is part of the control system 170. The user accounts 181 can include registrations and account data 182 and user profiles 183. The control system can also include a computer 172 having a computer readable storage medium 173 which can store programs or code embedded on the storage medium. The program code can be executed by a processor 175. The computer 172 can communicate with a database 176. The control system 170 can also include a database 176 for storing all or part of such data as described above, and other data.

The control system can also communicate with a computer system 190 which can include a learning engine/module 192 and a knowledge corpus or database 196. The computer system 190 can also communicate with the computer 131 of the device 130 and can be remote from the user device 130. In another example, the computer system 190 can be all or part of the control system, or all or part of the device 130. The depiction of the computer system 190 as well as the other components of the system 100 are shown as one example according to the present disclosure.

The new or different AI (Artificial Intelligence) ecosystem, or technology/communication or IT (Information Technology) ecosystem can include a local communications network 152 which can communicate with the communications network 160. The system 100 can include a learning engine/module 192, which can be at least part of the control system or communicating with the control system, for generating a model or learning model. In one example, the learning model can model workflow in a new AI or IT ecosystem for machine/devices in the new ecosystem.

In another example, the computer 131 can be part of a device 130. The computer can include a processor 132 and a computer readable storage medium 134 where an application 135 can be stored which can in one example, embody all or part of the method of the present disclosure. The application can include all or part of instructions to implement the method of the present disclosure, embodied in code and stored on a computer readable storage medium. The device 148 can include a display. The device 148 can operate, in all or in part, in conjunction with a remote server by way of a communications network 160, for example, the Internet.

A model can also be generated by an AI system such as an output at least in part of an AI system analysis using machine learning.

In one example, as part of the analysis of received data including data in the knowledge corpus and historical database 196, which can be populated by historical data gathered, for example, from sensors, robotic devices, or other machines.

### OTHER EMBODIMENTS AND EXAMPLES

Referring to FIG. 1, the device 130, also can be referred to as a user device or an administrator's device, includes a computer 131 having a processor 132 and a storage medium 134 where an application 135, can be stored. The application can embody the features of the method of the present disclosure as instructions. The user can connect to a learning engine 150 using the device 130. The device 130 which includes the computer 131 and a display or monitor 138. The application 135 can embody the method of the present disclosure and can be stored on the computer readable storage medium 134. The device 130 can further include the processor 132 for executing the application/software 135. The device 130 can communicate with a communications network 160, e.g., the Internet.

It is understood that the user device 130 is representative of similar devices which can be for other users, as representative of such devices, which can include, mobile devices, smart devices, laptop computers etc.

In one example, the system of the present disclosure can include a control system 170 communicating with the user device 130 via a communications network 160. The control system can incorporate all or part of an application or software for implementing the method of the present disclosure. The control system can include a computer readable storage medium 180 where account data and/or registration data 182 can be stored. User profiles 183 can be part of the account data and stored on the storage medium 180. The control system can include a computer 172 having computer readable storage medium 173 and software programs 174 stored therein. A processor 175 can be used to execute or implement the instructions of the software program. The control system can also include a database 176.

In another example and embodiment, profiles can be saved for entities such as users, participants, operators, human operators, or robotic devices. Such profiles can supply data regarding the user and history of deliveries for analysis. In one example, a user can register or create an account using the control system 170 which can include one or more profiles 183 as part of registration and/or account data 182. The registration can include profiles for each user having personalized data. For example, users can register using a website via their computer and GUI (Graphical User Interface) interface. The registration or account data 182 can include profiles 183 for an account 181 for each user. Such accounts can be stored on the control system 170, which can also use the database 176 for data storage. A user and a related account can refer to, for example, a person, or an entity, or a corporate entity, or a corporate department, or another machine such as an entity for automation such as a system using, in all or in part, artificial intelligence.

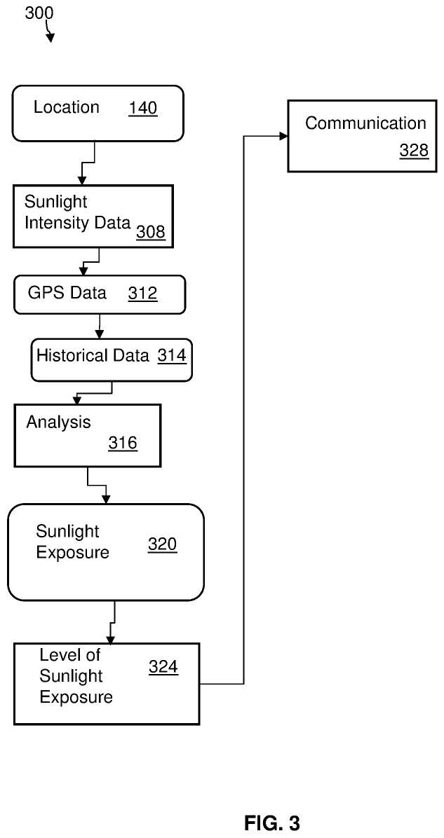

Additionally, the method and system is discussed with reference to FIG. 3, which is a functional system 300 which includes components and operations for embodiments according to the present disclosure, and is used herein for reference when describing the operational steps of the methods and systems of the present disclosure. Additionally, the functional system 300, according to an embodiment of the present disclosure, depicts functional operations indicative of the embodiments discussed herein.

Referring to FIG. 3, in one embodiment according to the present disclosure, a system 300 can be used to identify objects related to an event for use regarding the event by using networked computer system resources. In FIG. 3 similar components may have the same reference numerals as the system 100 shown in FIG. 1, the system 300 can include or operate in concert with a computer implemented method as shown in FIGS. 1 and 2.

### MORE EMBODIMENTS AND EXAMPLES

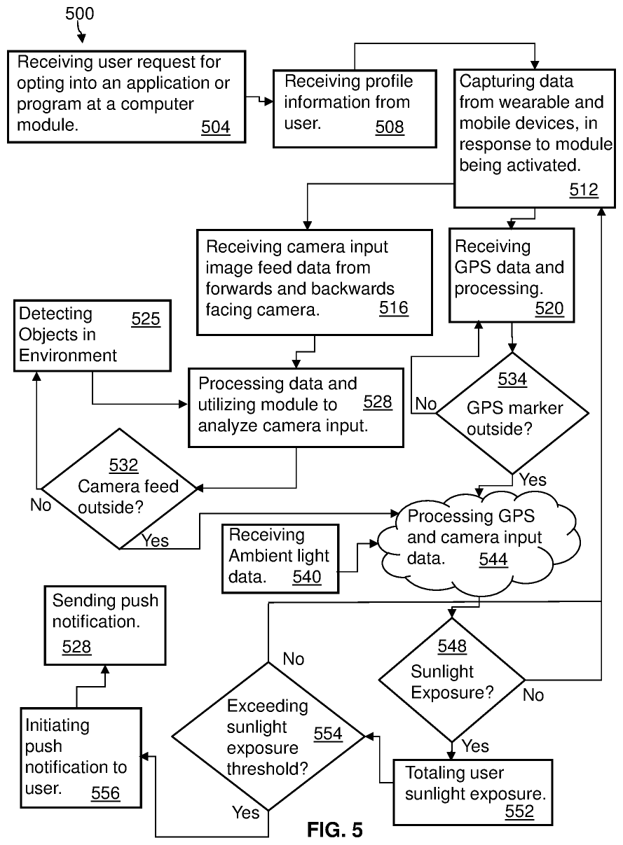

Generally referring to FIG. 5, in one embodiment according to the present disclosure, a system and method 500 can include receiving, at a computer, a user request for opting into an application or program in a computer module which incorporates in or is saved on a computer readable storage medium in a computer, as in block 504.

The method 500 includes receiving profile information from the user, as in block 508. The user can provide profile information including medical information or other relevant information. The method includes capturing data from wearable and mobile devices, in response to a module in the computer being activated, as in block 512. The method includes receiving camera input image feed data from a forward and backwards facing camera, as in block 516.

The method includes the module and computer processing data and utilizing the module to analyze the camera input, as in block 528. The method includes detecting object in the environment of a user at a location, as in block 525, and processing the data at block 528. Such object in the environment can include the sun, or artificial light, structural objects, etc. The method includes determining if a camera feed is capturing video from outside, in block 532. If no, the method returns to block 525. If yes, the method continues to block 544.

The method includes receiving GPS data and processing, as in block 520. The method further includes determining if the method has received GPS (Global Positioning System) data or markers in block 534. If no, the method returns to block 520. If yes, the method proceeds to block 544. The method includes receiving ambient light data, at block 540.

The method includes processing GPS and camera input data for determining if a user is outside by analyzing all the data including the data from the user's mobile phone, as in block 544, including the data from blocks 534, 540, and 532.

The method 500 includes determining whether the user is exposed to sunlight, at block 548. If no, the method returns to block 512. If yes, the method proceeds to totaling or counting the user's sunlight exposure, as in block 552. The method can include a threshold for sun exposure and determining when a threshold is reached or exceeded.

The method includes determining when a sunlight exposure threshold is exceeded, at block 554. If no, the method returns to block 512 to continue capturing or monitoring sunlight exposure in the environment using wearable or mobile devices. If yes, the method proceeds to initiating a push notification to the user, as in block 556. The method includes sending the push notification, as in block 528.

Thereby, in an embodiment according to the present disclosure, a system and method includes a user opting into the invention module. The user can provide a historical medical profile of themselves along with desired maximum exposure to sunlight in a given day. In one embodiment default timing can be used. In one embodiment the user can set one or more thresholds to activate the invention module such as sunlight through window counting towards sunlight exposure, user in a vehicle counting towards sunlight exposure, and a user in a mixed indoor/outdoor location (e.g., patio) counting towards sunlight exposure. In one example, invention module activates on a user's mobile device, such as a smartphone, smart watch, or a wearable. Activating can include capturing GPS location data, capturing image feed from forward and backwards facing camera, capturing ambient light sensor data and providing it to the invention module. An invention module can analyze at the GPS location and camera feed data to make a decision surrounding the immediate environment with the presence of sunlight. Camera decisioning can be derived on a convolutional neural net (CNN) of image recognition of data from the mobile device. The module and time counter will activate if the user's device detects a live feed from the forwards or backwards facing camera. The feed can be processed with the CNN to make a decision surrounding if the user and device is inside or outside with regards to light exposure. Additionally, components considered in the decision may include but are not be limited to: presence of sun in camera, outside vegetation such as trees, bushes, sidewalk, vehicles, ceiling, indoor walls, etc. In some embodiments the neural net may be trained on a training set of classified indoor and outdoor images to know if the user is inside or outside. A GPS location can be utilized for a flag to indicate if the user is inside or outside. The flag can be captured based on the user's GPS micro location and their location association on a map for a building, empty space, sidewalk, etc. In one embodiment, navigation information such as if the user uses the GPS in walk mode can be considered. In one embodiment, mobility data can be based on the GPS movement delta and movement speed. Based on level of confidence and capability of ambient light sensor, a system and method can augment the provided data with information such as shade. Each domain of the components captured above can count towards a timer of sunlight exposure. The sunlight exposure time will pull user medical and profile information to determine the maximum allowed exposure for a given outing or time chunk (e.g., day). Whenever the user passes the given threshold, the invention module reacts and provides notification to the user. In one example, a number of minutes before exposure threshold reached push notification can be initiated. A push notification or vibration alert can be initiated to recommend a user is covered up, such as wearing a hat, sunscreen etc. In one embodiment, the user can pause the module based on the user proactively preparing by taking proper precautions to sunlight exposure.

Thereby, embodiments of the present disclosure can use measurements from sunlight exposure metrics. Further, user provided data can be used to provide a more user specific experience or pull from feeds the user may have such as mobile camera, GPS, etc.

In one embodiment according to the present disclosure, a system and method can utilize GPS location, photosensitive sensors and the forward and backwards facing camera to derive sunlight exposure metrics and risks for a user. In one example, a system and method to read data from GPS, photosensitive sensors, and the onboard cameras of a mobile device to estimate sunlight exposure for a user. In one example, a personal corpus for the user can include a user's medical history and preferred sunlight exposure time to notify the user when they pass the exposure risk amount over a period of time. In another example, GPS data can be used to confirm whether or not a given user's geolocation is likely outside versus inside. In another example, sunlight exposure metrics and brightness can be affected by an ambient sensor's detection of an amount of time of natural light or light indicative of exposure. In another example, the module can utilize a forward and backwards facing camera to look for an ongoing feed that is indicative of one facet of the device being exposed and not in a state such as within a pocket or bag. In another example, a module will utilize machine learning such as a convolutional neural network to classify an image feed provided by the onboard cameras as inside or outside based on the presence of walls, features, sun, etc. In another example, a module can leverage data feeds from n number of devices such as a user's smart watch, tablet, other wearables and IOT devices to assist in the classification of sunlight exposure.

The embodiments of the present disclosure include a system and method that can use techniques for detecting sunlight exposure for a user with access to a personalized profile, and push a notification based on threshold to a mobile device of the user. In one example, GPS coordinates can be used for general determination whether a user device and thereby the user is outside or inside. In another example, a neural net classification of mobile device camera feed can be used to identify outside or inside objects, in determining location of the user and exposure to sunlight. In another example, ambient light can be weighted to determine shade or sunlight exposure of a user, using the mobile device. The present disclosure can use thermal sensors for detecting sunlight exposure of a user, and can capture multiple feeds to detect a higher fidelity of when determining sunlight exposure.

### MORE EXAMPLES AND EMBODIMENTS

Operational blocks and system components shown in one or more of the figures may be similar to operational blocks and system components in other figures. The diversity of operational blocks and system components depict example embodiments and aspects according to the present disclosure. For example, methods shown are intended as example embodiments which can include aspects/operations shown and discussed previously in the present disclosure, and in one example, continuing from a previous method shown in another flow chart.

### ADDITIONAL EXAMPLES AND EMBODIMENTS

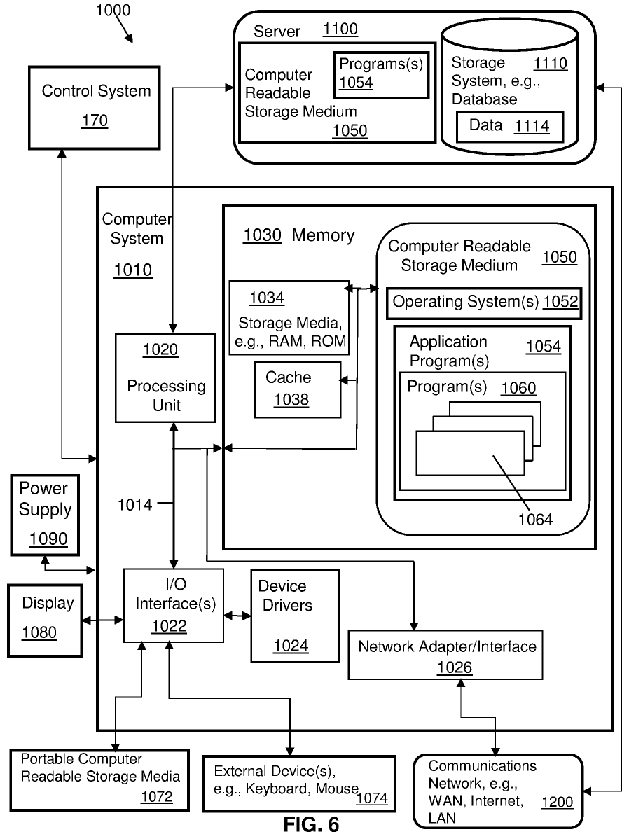

In the embodiment of the present disclosure shown in FIGS. 1 and 2, a computer can be part of a remote computer or a remote server, for example, remote server 1100 (FIG. 6). In another example, the computer 131 can be part of a control system 170 and provide execution of the functions of the present disclosure. In another embodiment, a computer can be part of a mobile device and provide execution of the functions of the present disclosure. In still another embodiment, parts of the execution of functions of the present disclosure can be shared between the control system computer and the mobile device computer, for example, the control system function as a back end of a program or programs embodying the present disclosure and the mobile device computer functioning as a front end of the program or programs.

The computer can be part of the mobile device, or a remote computer communicating with the mobile device. In another example, a mobile device and a remote computer can work in combination to implement the method of the present disclosure using stored program code or instructions to execute the features of the method(s) described herein. In one example, the device 130 can include a computer 131 having a processor 132 and a storage medium 134 which stores an application 135, and the computer includes a display 138. The application can incorporate program instructions for executing the features of the present disclosure using the processor 132. In another example, the mobile device application or computer software can have program instructions executable for a front end of a software application incorporating the features of the method of the present disclosure in program instructions, while a back end program or programs 174, of the software application, stored on the computer 172 of the control system 170 communicates with the mobile device computer and executes other features of the method. The control system 170 and the device (e.g., mobile device or computer) 130 can communicate using a communications network 160, for example, the Internet.

Thereby, the method 100 according to an embodiment of the present disclosure, can be incorporated in one or more computer programs or an application 135 stored on an electronic storage medium 134, and executable by the processor 132, as part of the computer on mobile device. For example, a mobile device can communicate with the control system 170, and in another example, a device such as a video feed device can communicate directly with the control system 170. Other users (not shown) may have similar mobile devices which communicate with the control system similarly. The application can be stored, all or in part, on a computer or a computer in a mobile device and at a control system communicating with the mobile device, for example, using the communications network 160, such as the Internet. It is envisioned that the application can access all or part of program instructions to implement the method of the present disclosure. The program or application can communicate with a remote computer system via a communications network 160 (e.g., the Internet) and access data, and cooperate with program(s) stored on the remote computer system. Such interactions and mechanisms are described in further detail herein and referred to regarding components of a computer system, such as computer readable storage media, which are shown in one embodiment in FIG. 6 and described in more detail in regards thereto referring to one or more computer systems 1010.

Thus, in one example, a control system 170 is in communication with the computer 131 or device 130, and the computer can include the application or software 135. The computer 131, or a computer in a mobile device 130 communicates with the control system 170 using the communications network 160.

In another example, the control system 170 can have a front-end computer belonging to one or more users, and a back-end computer embodied as the control system.

Also, referring to FIG. 1, a device 130 can include a computer 131, computer readable storage medium 134, and operating systems, and/or programs, and/or a software application 135, which can include program instructions executable using a processor 132. These features are shown herein in FIG. 1, and other similar components and features are also in an embodiment of a computer system shown in FIG. 6 referring to a computer system 1010, which may include one or more computer components.

The method according to the present disclosure, can include a computer for implementing the features of the method, according to the present disclosure, as part of a control system. In another example, a computer as part of a control system can work in corporation with a mobile device computer in concert with communication system for implementing the features of the method according to the present disclosure. In another example, a computer for implementing the features of the method can be part of a mobile device and thus implement the method locally.

Specifically, regarding the control system 170, a device(s) 130, or in one example devices which can belong to one or more users, can be in communication with the control system 170 via the communications network 160. In the embodiment of the control system shown in FIG. 1, the control system 170 includes a computer 172 communicating with a database 176 and one or more programs 174 stored on a computer readable storage medium 173. In the embodiment of the disclosure shown in FIG. 1, the device 130 communicates with the control system 170 and the one or more programs 174 stored on a computer readable storage medium 173. The control system includes the computer 172 having a processor 175, which also has access to the database 176.

The control system 170 can include a storage medium 180 for maintaining a registration 182 of users and their devices for analysis of the audio input. Such registration can include user profiles 183, which can include user data supplied by the users in reference to registering and setting-up an account. In an embodiment, the method and system which incorporates the present disclosure includes the control system (generally referred to as the back-end) in combination and cooperation with a front end of the method and system, which can be the application 135. In one example, the application 135 is stored on a device, for example, a computer or device on location, and can access data and additional programs at a back end of the application, e.g., control system 170.

The control system can also be part of a software application implementation, and/or represent a software application having a front-end user part and a back-end part providing functionality. In an embodiment, the method and system which incorporates the present disclosure includes the control system (which can be generally referred to as the back-end of the software application which incorporates a part of the method and system of an embodiment of the present application) in combination and cooperation with a front end of the software application incorporating another part of the method and system of the present application at the device, as in the example shown in FIG. 1 of a device 130 and computer 131 having the application 135. The application 135 is stored on the device or computer and can access data and additional programs at the back end of the application, for example, in the program(s) 174 stored in the control system 170.

The program(s) 174 can include, all or in part, a series of executable steps for implementing the method of the present disclosure. A program, incorporating the present method, can be all or in part stored in the computer readable storage medium on the control system or, in all or in part, on a computer or device 130. It is envisioned that the control system 170 can not only store the profile of users, but in one embodiment, can interact with a website for viewing on a display of a device such as a mobile device, or in another example the Internet, and receive user input related to the method and system of the present disclosure. It is understood that FIG. 1 depicts one or more profiles 183, however, the method can include multiple profiles, users, registrations, etc. It is envisioned that a plurality of users or a group of users can register and provide profiles using the control system for use according to the method and system of the present disclosure.

### STILL FURTHER EMBODIMENTS AND EXAMPLES

It is understood that the features shown in some of the FIGS., for example block diagrams, are functional representations of features of the present disclosure. Such features are shown in embodiments of the systems and methods of the present disclosure for illustrative purposes to clarify the functionality of features of the present disclosure.

The methods and systems of the present disclosure can include a series of operation blocks for implementing one or more embodiments according to the present disclosure. In some examples, operational blocks of one or more FIGS. may be similar to operational blocks shown in another figure. A method shown in one FIG. may be another example embodiment which can include aspects/operations shown in another FIG. and discussed previously.

### ADDITIONAL EMBODIMENTS AND EXAMPLES

Account data, for instance, including profile data related to a user, and any data, personal or otherwise, can be collected and stored, for example, in the control system 170. It is understood that such data collection is done with the knowledge and consent of a user, and stored to preserve privacy, which is discussed in more detail below. Such data can include personal data, and data regarding personal items.

In one example a user can register 182 have an account 181 with a user profile 183 on a control system 170, which is discussed in more detail below. For example, data can be collected using techniques as discussed above, for example, using cameras, and data can be uploaded to a user profile by the user. A user can include, for example, a corporate entity, or department of a business, or a homeowner, or any end user, a human operator, or a robotic device, or other personnel of a business.

Regarding collection of data with respect to the present disclosure, such uploading or generation of profiles is voluntary by the one or more users, and thus initiated by and with the approval of a user. Thereby, a user can opt-in to establishing an account having a profile according to the present disclosure. Similarly, data received by the system or inputted or received as an input is voluntary by one or more users, and thus initiated by and with the approval of the user. Thereby, a user can opt-in to input data according to the present disclosure. Such user approval also includes a user's option to cancel such profile or account, and/or input of data, and thus opt-out, at the user's discretion, of capturing communications and data. Further, any data stored or collected is understood to be intended to be securely stored and unavailable without authorization by the user, and not available to the public and/or unauthorized users. Such stored data is understood to be deleted at the request of the user and deleted in a secure manner. Also, any use of such stored data is understood to be, according to the present disclosure, only with the user's authorization and consent.

In one or more embodiments of the present invention, a user(s) can opt-in or register with a control system, voluntarily providing data and/or information in the process, with the user's consent and authorization, where the data is stored and used in the one or more methods of the present disclosure. Also, a user(s) can register one or more user electronic devices for use with the one or more methods and systems according to the present disclosure. As part of a registration, a user can also identify and authorize access to one or more activities or other systems (e.g., audio and/or video systems). Such opt-in of registration and authorizing collection and/or storage of data is voluntary and a user may request deletion of data (including a profile and/or profile data), un-registering, and/or opt-out of any registration. It is understood that such opting-out includes disposal of all data in a secure manner. A user interface can also allow a user or an individual to remove all their historical data.

### OTHER ADDITIONAL EMBODIMENTS AND EXAMPLES

In one example, Artificial Intelligence (AI) can be used, all or in part, for generating a model or a learning model as discussed herein in embodiments of the present disclosure.

An Artificial Intelligence (AI) System can include machines, computer, and computer programs which are designed to be intelligent or mirror intelligence. Such systems can include computers executing algorithms. AI can include machine learning and deep learning. For example, deep learning can include neural networks. An AI system can be cloud based, that is, using a cloud-based computing environment having computing resources.

In another example, the control system 170 can be all or part of an Artificial Intelligence (AI) system. For example, the control system can be one or more components of an AI system.

It is also understood that the method 100 according to an embodiment of the present disclosure, can be incorporated into (Artificial Intelligence) AI devices, components or be part of an AI system, which can communicate with respective AI systems and components, and respective AI system platforms. Thereby, such programs or an application incorporating the method of the present disclosure, as discussed above, can be part of an AI system. In one embodiment according to the present invention, it is envisioned that the control system can communicate with an AI system, or in another example can be part of an AI system. The control system can also represent a software application having a front-end user part and a back-end part providing functionality, which can in one or more examples, interact with, encompass, or be part of larger systems, such as an AI system. In one example, an AI device can be associated with an AI system, which can be all or in part, a control system and/or a content delivery system, and be remote from an AI device. Such an AI system can be represented by one or more servers storing programs on computer readable medium which can communicate with one or more AI devices. The AI system can communicate with the control system, and in one or more embodiments, the control system can be all or part of the AI system or vice versa.

It is understood that as discussed herein, a download or downloadable data can be initiated using a voice command or using a mouse, touch screen, etc. In such examples a mobile device can be user initiated, or an AI device can be used with consent and permission of users. Other examples of AI devices include devices which include a microphone, speaker, and can access a cellular network or mobile network, a communications network, or the Internet, for example, a vehicle having a computer and having cellular or satellite communications, or in another example, IoT (Internet of Things) devices, such as appliances, having cellular network or Internet access.

### FURTHER DISCUSSION REGARDING EXAMPLES AND EMBODIMENTS

It is understood that a set or group is a collection of distinct objects or elements. The objects or elements that make up a set or group can be anything, for example, numbers, letters of the alphabet, other sets, a number of people or users, and so on. It is further understood that a set or group can be one element, for example, one thing or a number, in other words, a set of one element, for example, one or more users or people or participants. It is also understood that machine and device are used interchangeable herein to refer to machine or devices in one or more AI ecosystems or environments.

The descriptions of the various embodiments of the present invention have been presented for purposes of illustration, but are not intended to be exhaustive or limited to the embodiments disclosed. Likewise, examples of features or functionality of the embodiments of the disclosure described herein, whether used in the description of a particular embodiment, or listed as examples, are not intended to limit the embodiments of the disclosure described herein, or limit the disclosure to the examples described herein. Such examples are intended to be examples or exemplary, and non-exhaustive. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### FURTHER ADDITIONAL EXAMPLES AND EMBODIMENTS

Referring to FIG. 6, an embodiment of system or computer environment 1000, according to the present disclosure, includes a computer system 1010 shown in the form of a generic computing device. The method 100, for example, may be embodied in a program 1060, including program instructions, embodied on a computer readable storage device, or a computer readable storage medium, for example, generally referred to as computer memory 1030 and more specifically, computer readable storage medium 1050. Such memory and/or computer readable storage media includes non-volatile memory or non-volatile storage, also known and referred to non-transient computer readable storage media, or non-transitory computer readable storage media. For example, such non-volatile memory can also be disk storage devices, including one or more hard drives. For example, memory 1030 can include storage media 1034 such as RAM (Random Access Memory) or ROM (Read Only Memory), and cache memory 1038. The program 1060 is executable by the processor 1020 of the computer system 1010 (to execute program steps, code, or program code). Additional data storage may also be embodied as a database 1110 which includes data 1114. The computer system 1010 and the program 1060 are generic representations of a computer and program that may be local to a user, or provided as a remote service (for example, as a cloud based service), and may be provided in further examples, using a website accessible using the communications network 1200 (e.g., interacting with a network, the Internet, or cloud services). It is understood that the computer system 1010 also generically represents herein a computer device or a computer included in a device, such as a laptop or desktop computer, etc., or one or more servers, alone or as part of a datacenter. The computer system can include a network adapter/interface 1026, and an input/output (I/O) interface(s) 1022. The I/O interface 1022 allows for input and output of data with an external device 1074 that may be connected to the computer system. The network adapter/interface 1026 may provide communications between the computer system a network generically shown as the communications network 1200.

The computer 1010 may be described in the general context of computer system-executable instructions, such as program modules, being executed by a computer system. Generally, program modules may include routines, programs, objects, components, logic, data structures, and so on that perform particular tasks or implement particular abstract data types. The method steps and system components and techniques may be embodied in modules of the program 1060 for performing the tasks of each of the steps of the method and system. The modules are generically represented in the figure as program modules 1064. The program 1060 and program modules 1064 can execute specific steps, routines, sub-routines, instructions or code, of the program.

The method of the present disclosure can be run locally on a device such as a mobile device, or can be run a service, for instance, on the server 1100 which may be remote and can be accessed using the communications network 1200. The program or executable instructions may also be offered as a service by a provider. The computer 1010 may be practiced in a distributed cloud computing environment where tasks are performed by remote processing devices that are linked through a communications network 1200. In a distributed cloud computing environment, program modules may be located in both local and remote computer system storage media including memory storage devices.

More specifically, the system or computer environment 1000 includes the computer system 1010 shown in the form of a general-purpose computing device with illustrative periphery devices. The components of the computer system 1010 may include, but are not limited to, one or more processors or processing units 1020, a system memory 1030, and a bus 1014 that couples various system components including system memory 1030 to processor 1020.

The bus 1014 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The computer 1010 can include a variety of computer readable media. Such media may be any available media that is accessible by the computer 1010 (e.g., computer system, or server), and can include both volatile and non-volatile media, as well as removable and non-removable media. Computer memory 1030 can include additional computer readable media in the form of volatile memory, such as random access memory (RAM) 1034, and/or cache memory 1038. The computer 1010 may further include other removable/non-removable, volatile/non-volatile computer storage media, in one example, portable computer readable storage media 1072. In one embodiment, the computer readable storage medium 1050 can be provided for reading from and writing to a non-removable, non-volatile magnetic media. The computer readable storage medium 1050 can be embodied, for example, as a hard drive. Additional memory and data storage can be provided, for example, as the storage system 1110 (e.g., a database) for storing data 1114 and communicating with the processing unit 1020. The database can be stored on or be part of a server 1100. Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1014 by one or more data media interfaces. As will be further depicted and described below, memory 1030 may include at least one program product which can include one or more program modules that are configured to carry out the functions of embodiments of the present invention.

The method(s) described in the present disclosure, for example, may be embodied in one or more computer programs, generically referred to as a program 1060 and can be stored in memory 1030 in the computer readable storage medium 1050. The program 1060 can include program modules 1064. The program modules 1064 can generally carry out functions and/or methodologies of embodiments of the invention as described herein. The one or more programs 1060 are stored in memory 1030 and are executable by the processing unit 1020. By way of example, the memory 1030 may store an operating system 1052, one or more application programs 1054, other program modules, and program data on the computer readable storage medium 1050. It is understood that the program 1060, and the operating system 1052 and the application program(s) 1054 stored on the computer readable storage medium 1050 are similarly executable by the processing unit 1020. It is also understood that the application 1054 and program(s) 1060 are shown generically, and can include all of, or be part of, one or more applications and program discussed in the present disclosure, or vice versa, that is, the application 1054 and program 1060 can be all or part of one or more applications or programs which are discussed in the present disclosure. It is also understood that a control system 170, communicating with a computer system, can include all or part of the computer system 1010 and its components, and/or the control system can communicate with all or part of the computer system 1010 and its components as a remote computer system, to achieve the control system functions described in the present disclosure. The control system function, for example, can include storing, processing, and executing software instructions to perform the functions of the present disclosure. It is also understood that the one or more computers or computer systems shown in FIG. 1 similarly can include all or part of the computer system 1010 and its components, and/or the one or more computers can communicate with all or part of the computer system 1010 and its components as a remote computer system, to achieve the computer functions described in the present disclosure.

In an embodiment according to the present disclosure, one or more programs can be stored in one or more computer readable storage media such that a program is embodied and/or encoded in a computer readable storage medium. In one example, the stored program can include program instructions for execution by a processor, or a computer system having a processor, to perform a method or cause the computer system to perform one or more functions. For example, in one embedment according to the present disclosure, a program embodying a method is embodied in, or encoded in, a computer readable storage medium, which includes and is defined as, a non-transient or non-transitory computer readable storage medium. Thus, embodiments or examples according to the present disclosure, of a computer readable storage medium do not include a signal, and embodiments can include one or more non-transient or non-transitory computer readable storage mediums. Thereby, in one example, a program can be recorded on a computer readable storage medium and become structurally and functionally interrelated to the medium.

The computer 1010 may also communicate with one or more external devices 1074 such as a keyboard, a pointing device, a display 1080, etc.; one or more devices that enable a user to interact with the computer 1010; and/or any devices (e.g., network card, modem, etc.) that enables the computer 1010 to communicate with one or more other computing devices. Such communication can occur via the Input/Output (I/O) interfaces 1022. A power supply 1090 can also connect to the computer using an electrical power supply interface (not shown). Still yet, the computer 1010 can communicate with one or more networks 1200 such as a local area network (LAN), a general wide area network (WAN), and/or a public network (e.g., the Internet) via network adapter/interface 1026. As depicted, network adapter 1026 communicates with the other components of the computer 1010 via bus 1014. It should be understood that although not shown, other hardware and/or software components could be used in conjunction with the computer 1010. Examples, include, but are not limited to: microcode, device drivers 1024, redundant processing units, external disk drive arrays, RAID systems, tape drives, and data archival storage systems, etc.

It is understood that a computer or a program running on the computer 1010 may communicate with a server, embodied as the server 1100, via one or more communications networks, embodied as the communications network 1200. The communications network 1200 may include transmission media and network links which include, for example, wireless, wired, or optical fiber, and routers, firewalls, switches, and gateway computers. The communications network may include connections, such as wire, wireless communication links, or fiber optic cables. A communications network may represent a worldwide collection of networks and gateways, such as the Internet, that use various protocols to communicate with one another, such as Lightweight Directory Access Protocol (LDAP), Transport Control Protocol/Internet Protocol (TCP/IP), Hypertext Transport Protocol (HTTP), Wireless Application Protocol (WAP), etc. A network may also include a number of different types of networks, such as, for example, an intranet, a local area network (LAN), or a wide area network (WAN).

In one example, a computer can use a network which may access a website on the Web (World Wide Web) using the Internet. In one embodiment, a computer 1010, including a mobile device, can use a communications system or network 1200 which can include the Internet, or a public switched telephone network (PSTN) for example, a cellular network. The PSTN may include telephone lines, fiber optic cables, microwave transmission links, cellular networks, and communications satellites. The Internet may facilitate numerous searching and texting techniques, for example, using a cell phone or laptop computer to send queries to search engines via text messages (SMS), Multimedia Messaging Service (MMS) (related to SMS), email, or a web browser. The search engine can retrieve search results, that is, links to websites, documents, or other downloadable data that correspond to the query, and similarly, provide the search results to the user via the device as, for example, a web page of search results.

### OTHER ASPECTS AND EXAMPLES

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures of the present disclosure illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### ADDITIONAL ASPECTS AND EXAMPLES

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

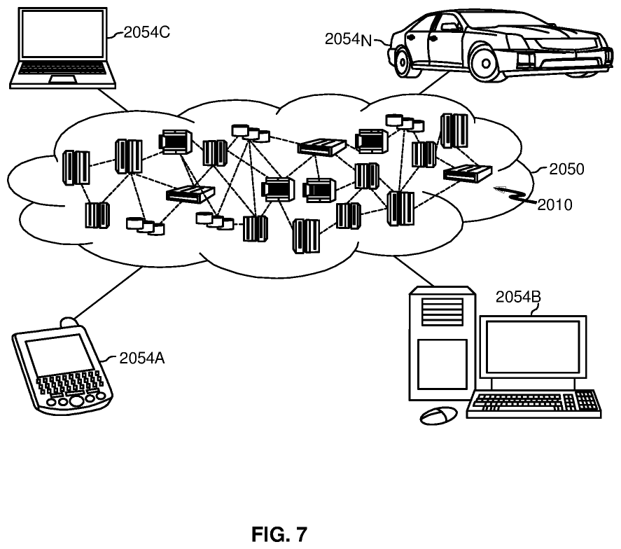

Referring now to FIG. 7, illustrative cloud computing environment 2050 is depicted. As shown, cloud computing environment 2050 includes one or more cloud computing nodes 2010 with which local computing devices used by cloud consumers, such as, for example, personal digital assistant (PDA) or cellular telephone 2054A, desktop computer 2054B, laptop computer 2054C, and/or automobile computer system 2054N may communicate. Nodes 2010 may communicate with one another. They may be grouped (not shown) physically or virtually, in one or more networks, such as Private, Community, Public, or Hybrid clouds as described hereinabove, or a combination thereof. This allows cloud computing environment 2050 to offer infrastructure, platforms and/or software as services for which a cloud consumer does not need to maintain resources on a local computing device. It is understood that the types of computing devices 2054A-N shown in FIG. 7 are intended to be illustrative only and that computing nodes 2010 and cloud computing environment 2050 can communicate with any type of computerized device over any type of network and/or network addressable connection (e.g., using a web browser).

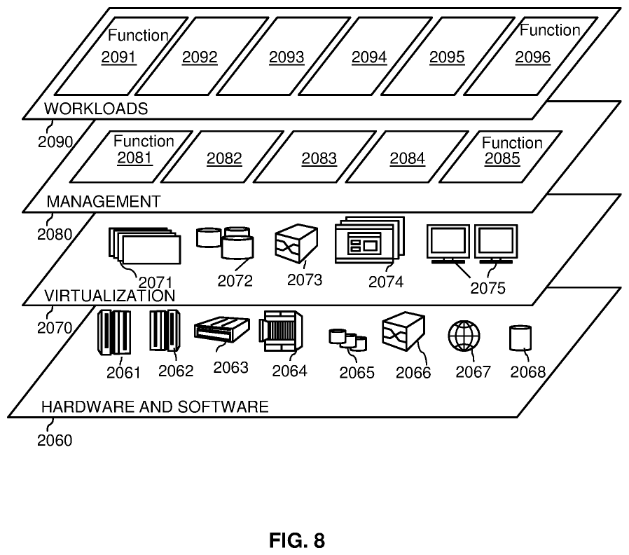

Referring now to FIG. 8, a set of functional abstraction layers provided by cloud computing environment 2050 (FIG. 7) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 2060 includes hardware and software components. Examples of hardware components include: mainframes 2061; RISC (Reduced Instruction Set Computer) architecture based servers 2062; servers 2063; blade servers 2064; storage devices 2065; and networks and networking components 2066. In some embodiments, software components include network application server software 2067 and database software 2068.

Virtualization layer 2070 provides an abstraction layer from which the following examples of virtual entities may be provided: virtual servers 2071; virtual storage 2072; virtual networks 2073, including virtual private networks; virtual applications and operating systems 2074; and virtual clients 2075.

In one example, management layer 2080 may provide the functions described below. Resource provisioning 2081 provides dynamic procurement of computing resources and other resources that are utilized to perform tasks within the cloud computing environment. Metering and Pricing 2082 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 2083 provides access to the cloud computing environment for consumers and system administrators. Service level management 2084 provides cloud computing resource allocation and management such that required service levels are met. Service Level Agreement (SLA) planning and fulfillment 2085 provide pre-arrangement for, and procurement of, cloud computing resources for which a future requirement is anticipated in accordance with an SLA.

Workloads layer 2090 provides examples of functionality for which the cloud computing environment may be utilized. Examples of workloads and functions which may be provided from this layer include: mapping and navigation 2091; software development and lifecycle management 2092; virtual classroom education delivery 2093; data analytics processing 2094; transaction processing 2095; and analyzing environmental data for generating a notification using a mobile device 2096.

What is claimed is:

1. A computer-implemented method using a mobile device for detecting and communicating to a user at a location, sunlight exposure in relation to the user at the location, comprising:
determining, at a computer, sunlight intensity data at a location of a mobile device, in response to receiving at the computer, GPS (Global Positioning System) data for the mobile device;
analyzing the sunlight intensity data and the GPS data to generate an estimate of sunlight exposure for a user using the mobile device at the location;
determining a level of sunlight exposure for the user, based on the estimate of sunlight exposure for the user; and
communicating, using the mobile device, the level of sunlight exposure to the user.

2. The method of claim 1, wherein the communicating includes sending a notification to the mobile device communicating the level of sunlight exposure to the user.

3. The method of claim 1, wherein the communicating includes displaying the level of sunlight exposure on a display of the mobile device.

4. The method of claim 1, wherein the communicating includes generating an audible notification of the level of sunlight exposure using the mobile device.

5. The method of claim 1, wherein the sunlight intensity data is determined using a camera of the mobile device to estimate the sunlight intensity.

6. The method of claim 1, wherein the computer is part of the mobile device.

7. The method of claim 1, wherein the computer is at least part of a remote device communicating with the mobile device.

8. The method of claim 1, further comprising:
determining a sunlight exposure threshold for the user based on an exposure formula for safety; and
using the analysis of the sunlight intensity, determining when the sunlight exposure threshold is met, and in response to the sunlight exposure being met, communicating a warning to the user which include the communicating of the level of sunlight exposure.

9. The method of claim 1, further comprising:
accessing a historical data corpus for the user which includes a medical history;
determining a sunlight exposure threshold for the user based on the medical history;
using the analysis of the sunlight intensity data, determining when the sunlight exposure threshold is met; and
communicating an alert to the user which includes the communicating of the level of sunlight exposure, in response to the sunlight exposure being met.

10. The method of claim 1, further comprising:
generating a model, using the computer wherein the model includes the following;
updating the sunlight intensity data;
updating the analyzing of the sunlight intensity data;
updating the determining of the level of the sunlight exposure; and
updating the communicating of the level of the sunlight exposure.

11. The method of claim 10, further comprising:
iteratively generating the model to produce updated models.

12. A system for method using a mobile device for detecting and communicating to a user at a location, sunlight exposure in relation to the user at the location, which comprises:
a computer system comprising; a computer processor, a computer-readable storage medium, and program instructions stored on the computer-readable storage medium being executable by the processor, to cause the computer system to perform the following functions to;
determining, at a computer, sunlight intensity data at a location of a mobile device, in response to receiving at the computer, GPS (Global Positioning System) data for the mobile device;
analyzing the sunlight intensity data and the GPS data to generate an estimate of sunlight exposure for a user using the mobile device at the location;
determining a level of sunlight exposure for the user, based on the estimate of sunlight exposure for the user; and
communicating, using the mobile device, the level of sunlight exposure to the user.

13. The system of claim 12, wherein the communicating includes sending a notification to the mobile device communicating the level of sunlight exposure to the user.

14. The system of claim 12, wherein the communicating includes displaying the level of sunlight exposure on a display of the mobile device.

15. The system of claim 12, wherein the communicating includes generating an audible notification of the level of sunlight exposure using the mobile device.

16. The system of claim 12, wherein the sunlight intensity data is determined using a camera of the mobile device to estimate the sunlight intensity.

17. The system of claim 12, wherein the computer is part of the mobile device.

18. The system of claim 12, wherein the computer is at least part of a remote device communicating with the mobile device.

19. The system of claim 12, further comprising:
determining a sunlight exposure threshold for the user based on an exposure formula for safety; and
using the analysis of the sunlight intensity, determining when the sunlight exposure threshold is met, and in response to the sunlight exposure being met, communicating a warning to the user which include the communicating of the level of sunlight exposure.

20. A computer program product for method using a mobile device for detecting and communicating to a user at a location, sunlight exposure in relation to the user at the location, the computer program product comprising a computer readable storage medium having program instructions embodied therewith, the program instructions executable by a computer to cause the computer to perform functions, by the computer, comprising the functions to;
determining, at a computer, sunlight intensity data at a location of a mobile device, in response to receiving at the computer, GPS (Global Positioning System) data for the mobile device;
analyzing the sunlight intensity data and the GPS data to generate an estimate of sunlight exposure for a user using the mobile device at the location;
determining a level of sunlight exposure for the user, based on the estimate of sunlight exposure for the user; and
communicating, using the mobile device, the level of sunlight exposure to the user.
